(19) United States
(12) Patent Application Publication
(10) Pub. No.: US 2005/0223441 A1
(43) Pub. Date: Oct. 6, 2005

(54) Methods for increasing the amount of protein in potato tubers

(76) Inventors: Clarence A. Ryan, Pullman, WA (US); Julian Narvaez, Riverside, CA (US); Gregory L. Pearce, Palouse, WA (US); Barry F. McGurl, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/505,190

(22) PCT Filed: Feb. 20, 2003
(86) PCT No.: PCT/US03/05026

§ 371 Date: May 10, 2005

Related U.S. Application Data
(60) Provisional application No. 60/359,472, filed on Feb. 22, 2002.

(51) Int. Cl. A01H 5/00; A01H 1/00; C12N 15/82

(52) U.S. Cl. 800/317.2; 800/298; 800/290

(57) ABSTRACT

In one aspect, the present invention provides potato tubers that each include water-soluble protein at a concentration of at least 5 mg/ml. Some potato tubers of the invention include an exogenous nucleic acid molecule that encodes a systemin molecule, or a prosystemin molecule, that is expressed in the potato tubers, whereby the synthesis of at least one water-soluble protein is induced by the expressed systemin molecule (or the systemin molecule derived from processed prosystemin). In another aspect, the present invention provides methods for increasing the amount of water-soluble protein in a potato tuber.

### FIELD OF THE INVENTION

The present invention relates to methods for increasing the amount of protein in potato tubers, and to potato tubers having elevated levels of protein.

### BACKGROUND OF THE INVENTION

The tubers of the potato plant (Solanum tuberosum) are an important source of nutrition for the human population. In addition to carbohydrates, potato tubers provide a source of protein in the diet. For example, proteinase inhibitor I and proteinase inhibitor II are abundant, water-soluble, storage proteins that are present in potato tubers, where their levels have been shown to closely correlate with the amount of total protein in the tubers (Ryan et al., Am. Potato J. 53: 443-455 (1976); Ryan and Pearce, Am. Potato J. 55: 351-358 (1978)). The proteins denature readily when cooked, and become excellent food proteins, being high in lysine and sulfur-containing amino acids.

Proteinase inhibitor I and proteinase inhibitor II are also inducibly expressed in the leaves of potato plants (and in the leaves of taxonomically-related tomato plants) in response to wounding, such as physical damage caused by insects feeding on the plant (Green, T. and Ryan, C. A., Science 175: 776-777 (1972); Pearce, G. L., et al., Proc. Soc. Expt'L. Biol. Med, 160: 180-184 (1979)). Wound-inducible expression of proteinase inhibitor I and proteinase inhibitor II in tomato plant leaves has been extensively investigated and is mediated by a mobile, eighteen amino acid, polypeptide signaling molecule called systemin (Reviewed by C. A. Ryan, Biochimica et Biophysica Acta 1477: 112-121 (2000)). Systemin is believed to be released from damaged cells at the site of wounding, and to travel within the phloem to unwounded parts of the plant where systemin induces synthesis of at least eighteen proteins, including proteinase inhibitors I and II. Some of the induced proteins are involved in plant defense and deter further predation by insects or pathogens.

Systemin is synthesized as part of a larger precursor molecule called prosystemin. A cDNA and gene encoding prosystemin have been isolated from tomato plants (McGurl et al., Science 255: 1570-1573 (1992)). cDNA clones encoding prosystemin molecules have also been isolated from potato, black nightshade and bell pepper, and the amino acid sequence of mature systemin in each of these species has been deduced from the prosystemin sequence (see, C. A. Ryan, Biochimica et Biophysica Acta 1477: 112-121 (2000)). Transgenic tomato plants that overexpress tomato prosystemin constitutively express high levels of proteinase inhibitors I and II in their leaves (McGurl et al., Proc.

Nat'l Acad. Sci. U.S.A., 91: 9799-9802 (1994)), and transgenic tomato plants that constitutively express a prosystemin antisense cDNA molecule produce lower amounts of proteinase inhibitors I and II in their leaves in response to wounding compared to control tomato plants that do not express a prosystemin antisense cDNA molecule (McGurl et al., Science 255: 1570-1573 (1992)).

Proteinase inhibitors I and II are also developmentally expressed in tomato flowers, and in potato tubers, during the development of these organs. To the best of applicants' knowledge, there is no evidence in the literature to suggest that systemin has a role in regulating the developmental expression of proteinase inhibitors I and II in either tomato flowers or potato tubers.

The present inventors have unexpectedly found that constitutive expression of a tomato prosystemin polypeptide in potato plants increases the amount of water-soluble protein in potato tubers. In particular, the present inventors have unexpectedly found that the amounts of proteinase inhibitor I, proteinase inhibitor II and patatin (another storage protein found in potato tubers) are increased in potato tubers obtained from transgenic potato plants that express a tomato prosystemin polypeptide.

### SUMMARY OF THE INVENTION

In one aspect, the present invention provides potato tubers that each include water-soluble protein at a concentration of at least 5 mg/ml (such as at least 7 mg/ml, or such as at least 10 mg/ml, or such as at least 20 mg/ml). Some potato tubers of the invention include water-soluble protein at a concentration of at least 5 mg/ml (such as at least 7 mg/ml, or such as at least 10 mg/ml, or such as at least 20 mg/ml), proteinase inhibitor I at a concentration of at least 500 μg/ml (such as at least 750 μg/ml, or such as at least 1000 μg/ml), and proteinase inhibitor II at a concentration of at least 800 μg/ml (such as at least 1000 μg/ml, or such as at least 1200 μg/ml).

Some potato tubers of the invention include water-soluble protein at a concentration of at least 5 mg/ml (such as at least 7 mg/ml, or such as at least 10 mg/ml, or such as at least 20 mg/ml), and also include an exogenous nucleic acid molecule that encodes a systemin molecule, or a prosystemin molecule, that is expressed in the potato tubers, whereby the synthesis of at least one water-soluble protein is induced by the expressed systemin molecule (or the systemin molecule derived from processed prosystemin). Exemplary systemin molecules useful in the practice of the invention have an amino acid sequence that is at least 70% identical (such as at least 80% identical, or such as at least 90% identical, or such as at least 95% identical, or such as at least 99% identical) to the tomato systemin amino acid sequence set forth in SEQ ID NO:1. An exemplary prosystemin molecule useful in the practice of the invention is encoded by the cDNA molecule having the nucleic acid sequence set forth in SEQ ID NO:2, and has the amino acid sequence set forth in SEQ ID NO:3.

The present invention also provides potato tubers that each include an exogenous nucleic acid molecule that encodes a systemin molecule. Some potato tubers of the invention include an exogenous nucleic acid molecule that (a) encodes a systemin molecule, and (b) hybridizes under conditions of 5×SSC at 55° C. for 30 minutes to a nucleic acid molecule consisting of the nucleic acid sequence set forth in SEQ ID NO:4. Some potato tubers of the invention include an exogenous nucleic acid molecule that (a) encodes a systernin molecule, and (b) hybridizes under conditions of 1×SSC at 50° C. for 30 minutes to the complement of a nucleic acid molecule consisting of the nucleic acid sequence set forth in SEQ ID NO:2.

The present invention also provides potato tubers that each include an exogenous nucleic acid molecule that encodes a prosystemin molecule, wherein the nucleic acid molecule hybridizes under conditions of 1×SSC at 50° C. for 30 minutes to the complement of a nucleic acid molecule consisting of the nucleic acid sequence set forth in SEQ ID NO:2.

In another aspect, the present invention provides methods for increasing the amount of water-soluble protein in a potato tuber. The methods of this aspect of the invention each include the step of expressing an exogenous systemin molecule in the potato tuber, whereby the synthesis of at least one water-soluble protein is induced by the exogenous systemin molecule. The exogenous systemin molecule can be expressed as a mature eighteen amino acid polypeptide, or can be expressed as a larger precursor protein, which is processed within a potato cell to yield a mature, eighteen amino acid, systemin polypeptide.

The potato tubers and methods of the invention are useful in any situation in which potato tubers having elevated levels of water-soluble protein (and/or elevated levels of proteinase inhibitor I, or proteinase inhibitor II, or patatin) are desired. For example, the potato tubers of the invention are a protein-rich foodstuff, and are of particular value to people in economically underdeveloped countries where sufficient dietary protein is difficult to obtain. The potato tubers of the invention are a protein-rich foodstuff that can be fed to animals to enhance their growth rate and meat quality. Also, the potato tubers of the invention can be used as a source from which to isolate proteinase inhibitor I and/or proteinase inhibitor II, which have a variety of uses, such as use as a dietary supplement that delays or prevents the onset of adult-onset diabetes. The potato tubers of the invention are also useful in any situation in which potato tubers that do not have elevated levels of water-soluble protein are useful. Thus, for example, the potato tubers of the invention can be used to make potato chips and other edible, processed, potato products.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows the amount of proteinase inhibitor I protein plotted against the amount of water-soluble protein in juice expressed from tubers of transgenic potato plants (filled diamonds) and from tubers of wild type control potato plants (filled squares).

FIG. 2 shows the amount of proteinase inhibitor II protein plotted against the amount of water-soluble protein in juice expressed from tubers of transgenic potato plants (filled diamonds) and from tubers of wild type control potato plants (filled squares).

### DETAILED DESCRIPTION OF THE PREFERRED EMBODIMENT

Unless specifically defined herein, all terms used herein have the same meaning as they would to one skilled in the art of the present invention. Practitioners are particularly directed to Sambrook et al. (1989) Molecular Cloning: A Laboratory Manual, 2^{nd} ed., Cold Spring Harbor Press, Plainsview, N.Y. (1989), and Ausubel et al., Current Protocols in Molecular Biology (Supplement 47), John Wiley & Sons, New York (1999), for definitions and terms of the art. Unless stated otherwise, all publications and patents that are cited in the present patent application are incorporated herein by reference in their entirety.

As used herein, the term “systemin” refers to a polypeptide that is capable of inducing the synthesis of proteinase inhibitor I and/or proteinase inhibitor II in a potato tuber, and which is at least 50% identical to the tomato systemin polypeptide consisting of the amino acid sequence set forth in SEQ ID NO:1.

As used herein, the term “prosystemin” means a polypeptide precursor that is degraded in vivo to yield a systemin, and which is at least 50% identical to the tomato prosystemin polypeptide consisting of the amino acid sequence set forth in SEQ ID NO:3.

As used herein, the term “potato tuber” refers to a tuber of the potato plant Solanum tuberosum.

As used herein, the term “exogenous nucleic acid molecule” means a nucleic acid molecule that has been introduced into a potato tuber by human activity. For example, a nucleic acid molecule that encodes a systemin molecule can be incorporated into a vector, and then introduced into a potato plant, by art-recognized means, to yield a genetically-modified potato plant wherein the exogenous nucleic acid molecule, that encodes a systemin molecule, is present in all cells of the genetically-modified potato plant, including cells of potato tubers grown from the genetically-modified potato plant.

As used herein, the term “exogenous systemin molecule” means a systemin molecule that has been introduced into a potato tuber by human activity. For example, a nucleic acid molecule that encodes a systemin molecule can be incorporated into a vector, and then introduced into a potato plant, by art-recognized means, to yield a genetically-modified potato plant wherein the exogenous nucleic acid molecule, that encodes a systemin molecule, is present in all cells of the genetically-modified potato plant, including cells of potato tubers grown from the genetically-modified potato plant. The exogenous nucleic acid molecule may express the systemin in cells of the potato tubers. The expressed systemin molecule is an exogenous systemin molecule.

As used herein, the term “percent identity” or “percent identical”, when used in connection with systemin molecules useful in the practice of the present invention, is defined as the percentage of amino acid residues in a systemin molecule sequence that are identical with the amino acid sequence of a specified systemin molecule (such as the amino acid sequence of SEQ ID NO:1), after aligning the systemin sequences to achieve the maximum percent identity. When making the comparison, no gaps are introduced into the systemin sequences in order to achieve the best alignment.

Amino acid sequence identity can be determined, for example, in the following manner. The amino acid sequence of a systemin molecule is used to search a protein sequence database, such as the GenBank database (accessible at web site http://www.ncbi.nln.nih.gov/blast/), using the BLASTP program. The program is used in the ungapped mode. Default filtering is used to remove sequence homologies due to regions of low complexity. The default parameters of BLASTP are utilized. Additionally, because systemin molecules are short, amino acid sequence identity between two systemin molecules can be determined, for example, in the following manner. The amino acid sequences of two systemin molecules are written on separate strips of paper which are then aligned and moved past each other until the maximum sequence alignment is achieved.

The abbreviation “SSC” refers to a buffer used in nucleic acid hybridization solutions. One liter of the 20× (twenty times concentrate) stock SSC buffer solution (pH 7.0) contains 175.3 g sodium chloride and 88.2 g sodium citrate.

In one aspect, the present invention provides potato tubers that each include water-soluble protein at a concentration of at least 5 mg/ml. Some potato tubers of this aspect of the invention include water-soluble protein at a concentration of at least 7 mg/ml. Some potato tubers of this aspect of the invention include water-soluble protein at a concentration of at least 10 mg/ml. Some potato tubers of this aspect of the invention include water-soluble protein at a concentration of at least 20 mg/ml.

Some potato tubers of this aspect of the invention include water-soluble protein at a concentration of from 5 mg/ml to 20 mg/ml. Some potato tubers of this aspect of the invention include water-soluble protein at a concentration of from 7 mg/ml to 15 mg/ml. Some potato tubers of this aspect of the invention include water-soluble protein at a concentration of from 7 mg/ml to 10 mg/ml.

The concentration of water-soluble protein is the total concentration of all of the water-soluble proteins in the tuber. The concentration of water-soluble protein can be measured as follows: a transverse section (about one quarter inch in width) is excised from the center of a potato tuber, including cortical and pith tissue. The section is diced into small pieces and crushed with mortar and pestle to express the juice. The juice is collected in a microfuge tube and centrifuged at 8000 rpm to clarify. 10 μl of clarified juice are added to 3 ml of Bradford reagent prepared in accordance with the manufacturer's instructions (Bradford reagent is sold by BioRad, Hercules, Calif.). The expressed juice is mixed with the Bradford reagent and the absorbance at 595 nm is recorded. The recorded absorbance value is compared to a standard curve of protein concentration to determine the amount of water-soluble protein in the juice. The standard curve is constructed using Bovine Serum Albumin as the standard.

Some potato tubers of the invention include water-soluble protein at a concentration of at least 5 mg/ml, proteinase inhibitor I at a concentration of at least 500 μg/ml, and proteinase inhibitor II at a concentration of at least 800 μg/ml. Some potato tubers of the invention include water-soluble protein at a concentration of at least 7 mg/ml, proteinase inhibitor I at a concentration of at least 750 μg/ml, and proteinase Inhibitor II at a concentration of at least 1000 μg/ml. Some potato tubers of the invention include water-soluble protein at a concentration of at least 10 mg/ml, proteinase inhibitor I at a concentration of at least 1000 μg/ml, and proteinase Inhibitor II at a concentration of at least 1200 μg/ml. Some potato tubers of the invention include water-soluble protein at a concentration of at least 20 mg/ml, proteinase inhibitor I at a concentration of at least 500 μg/ml, and proteinase inhibitor II at a concentration of at least 800 μg/ml. The concentrations of proteinase inhibitor I and proteinase Inhibitor II can each be determined using the assay described in Example 3 herein.

Some potato tubers of the invention include an exogenous nucleic acid molecule that encodes a systemin molecule. The exogenous nucleic acid molecule is capable of expressing the encoded systemin molecule in one or more cell types of potato plants, such as the cells of potato tubers. It has been unexpectedly discovered by the present inventors that systemin molecules are capable of increasing the level of expression of proteinase inhibitor I, proteinase Inhibitor II and patatin (a potato tuber storage protein) in potato tubers. The exogenous nucleic acid molecule can encode a mature, eighteen amino acid, systemin molecule, or a prosystemin molecule that is processed in potato cells to yield a mature, eighteen amino acid, systemin molecule.

Systemin molecules, that are useful in the practice of the present invention, have an amino acid sequence that is at least 50% identical (such as at least 60% identical, or such as at least 70% identical, or such as at least 80% identical, or such as at least 90% identical, or such as at least 95% identical, or such as at least 99% identical) to the amino acid sequence set forth in SEQ ID NO:1. The amino acid sequence set forth in SEQ ID NO:1 is the sequence of a tomato systemin molecule.

Examples of systemin molecules useful in the practice of the present invention are disclosed in U.S. Pat. No. 6,022,739 which is incorporated herein by reference.

Other representative systemin molecules, that are useful in the practice of the present invention to increase the amount of water-soluble protein in potato tubers, are described in C. P. Constabel, et al., Plant Mol. Biol., 34: 55-62 (1998), which publication is incorporated herein by reference, and include the following systemin molecules: AVHSTPPSKRDPPKMQTD (SEQ ID NO:5) from potato; AAHSTPPSKRDPPKMQTD (SEQ ID NO:6) from potato; AVRSTPPPKRDPPKMQTD (SEQ ID NO:7) from nightshade; AVHSTPPSKRPPPKMQTD (SEQ ID NO:8) from pepper.

Thus, in one embodiment, the present invention provides potato tubers that each include water-soluble protein at a concentration of at least 5 mg/ml (such as at least 7 mg/ml, or such as at least 10 mg/ml, or such as at least 20 mg/ml), and wherein the potato tubers each include an exogenous nucleic acid molecule that encodes a systemin molecule, wherein the systemin molecule is at least 50% identical (such as at least 60% identical, or such as at least 70% identical, or such as at least 80% identical, or such as at least 90% identical, or such as at least 95% identical, or such as at least 99% identical) to the systemin molecule having the amino acid sequence set forth in SEQ ID NO:1.

Some potato tubers of the invention include an exogenous nucleic acid molecule that (a) encodes a systemin molecule, and (b) hybridizes to the following nucleic acid molecule under stringent conditions: 5′-GTCTGTTTGCATTTTGGGAGGATCACGCTTTGATGGAGGTTTTGATTGAACAG C-3′ (SEQ ID NO:4).

Thus, in one aspect, the present invention provides potato tubers that each include water-soluble protein at a concentration of at least 5 mg/ml (such as at least 7 mg/ml, or such as at least 10 mg/ml, or such as at least 20 mg/ml), and that each include an exogenous nucleic acid molecule that (a) encodes a systemin molecule, and (b) hybridizes to a nucleic acid molecule consisting of the nucleic acid sequence set forth in SEQ ID NO:4 under conditions of 5×SSC at 55° C. for 30 minutes. Some potato tubers of this aspect of the invention each include water-soluble protein at a concentration of at least 5 mg/ml (such as at least 7 mg/ml, or such as at least 10 mg/ml, or such as at least 20 mg/ml), and each include an exogenous nucleic acid molecule that (a) encodes a systemin molecule, and (b) hybridizes to a nucleic acid molecule consisting of the nucleic acid sequence set forth in SEQ ID NO:4 under conditions of 5×SSC at 60° C. for 30 minutes.

In another aspect, the present invention provides potato tubers that each include water-soluble protein at a concentration of at least 5 mg/ml (such as at least 7 mg/ml, or such as at least 10 mg/ml, or such as at least 20 mg/ml), proteinase inhibitor I at a concentration of at least 500 μg/ml (such as at least 750 μg/ml, or such as at least 1000 μg/ml), proteinase inhibitor II at a concentration of at least 800 μg/ml (such as at least 1000 μg/ml, or such as at least 1200 μg/ml), and that each include an exogenous nucleic acid molecule that (a) encodes a systemin molecule, and (b) hybridizes to a nucleic acid molecule consisting of the nucleic acid sequence set forth in SEQ ID NO:4 under conditions of 5×SSC at 55° C. for 30 minutes.

Some potato tubers of this aspect of the invention each include water-soluble protein at a concentration of at least 5 mg/ml (such as at least 7 mg/ml, or such as at least 10 mg/ml, or such as at least 20 mg/ml), proteinase inhibitor I at a concentration of at least 500 μg/ml (such as at least 750 μg/ml, or such as at least 1000 μg/ml), proteinase inhibitor II at a concentration of at least 800 μg/ml (such as at least 1000 μg/ml, or such as at least 1200 μg/ml), and each include an exogenous nucleic acid molecule that (a) encodes a systemin molecule, and (b) hybridizes to a nucleic acid molecule consisting of the nucleic acid sequence set forth in SEQ ID NO:4 under conditions of 5×SSC at 60° C. for 30 minutes.

Some potato tubers of the invention include an exogenous nucleic acid molecule that (a) encodes a systemin molecule, and (b) hybridizes to the complement of the nucleic acid molecule set forth in SEQ ID NO:2 under stringent conditions. Thus, in one aspect, the present invention provides potato tubers that each include water-soluble protein at a concentration of at least 5 mg/ml (such as at least 7 mg/ml, or such as at least 10 mg/ml, or such as at least 20 mg/ml), and that each include an exogenous nucleic acid molecule that (a) encodes a systemin molecule, and (b) hybridizes to the complement of the nucleic acid molecule set forth in SEQ ID NO:2 under conditions of 1×SSC at 50° C. for 30 minutes. Some potato tubers of this aspect of the invention each include water-soluble protein at a concentration of at least 5 mg/ml (such as at least 7 mg/ml, or such as at least 10 mg/ml, or such as at least 20 mg/ml), and each include an exogenous nucleic acid molecule that (a) encodes a systemin molecule, and (b) hybridizes to the complement of the nucleic acid molecule set forth in SEQ ID NO:2 under conditions of 0.5×SSC at 50° C. for 30 minutes. Some potato tubers of this aspect of the invention each include water-soluble protein at a concentration of at least 5 mg/ml (such as at least 7 mg/ml, or such as at least 10 mg/ml, or such as at least 20 mg/ml), and each include an exogenous nucleic acid molecule that (a) encodes a systemin molecule, and (b) hybridizes to the complement of the nucleic acid molecule set forth in SEQ ID NO:2 under conditions of 0.5×SSC at 60° C. for 30 minutes.

In another aspect, the present invention provides potato tubers that each include water-soluble protein at a concentration of at least 5 mg/ml (such as at least 7 mg/ml, or such as at least 10 mg/ml, or such as at least 20 mg/ml), proteinase inhibitor I at a concentration of at least 500 μg/ml (such as at least 750 μg/ml, or such as at least 1000 μg/ml), proteinase inhibitor II at a concentration of at least 800 μg/ml (such as at least 1000 μg/ml, or such as at least 1200 μg/ml), and that each include an exogenous nucleic acid molecule that (a) encodes a systemin molecule, and (b) hybridizes to the complement of the nucleic acid molecule set forth in SEQ ID NO:2 under conditions of 1×SSC at 50° C. for 30 minutes. Some potato tubers of this aspect of the invention each include water-soluble protein at a concentration of at least 5 mg/ml (such as at least 7 mg/ml, or such as at least 10 mg/ml, or such as at least 20 mg/ml), proteinase inhibitor I at a concentration of at least 500 μg/ml (such as at least 750 μg/ml, or such as at least 1000 μg/ml), proteinase inhibitor II at a concentration of at least 800 μg/ml (such as at least 1000 μg/ml, or such as at least 1200 μg/ml), and each include an exogenous nucleic acid molecule that (a) encodes a systemin molecule, and (b) hybridizes to the complement of the nucleic acid molecule set forth in SEQ ID NO:2 under conditions of 0.5×SSC at 50° C. for 30 minutes. Some potato tubers of this aspect of the invention each include water-soluble protein at a concentration of at least 5 mg/ml (such as at least 7 mg/ml, or such as at least 10 mg/ml, or such as at least 20 mg/ml), proteinase inhibitor I at a concentration of at least 500 μg/ml (such as at least 750 μg/ml, or such as at least 1000 μg/ml), proteinase inhibitor II at a concentration of at least 800 μg/ml (such as at least 1000 μg/ml, or such as at least 1200 μg/ml), and each include an exogenous nucleic acid molecule that (a) encodes a systemin molecule, and (b) hybridizes to the complement of the nucleic acid molecule set forth in SEQ ID NO:2 under conditions of 0.5×SSC at 60° C. for 30 minutes.

Some potato tubers of the invention include an exogenous nucleic acid molecule that (a) encodes a prosystemin molecule, and (b) hybridizes to the complement of the nucleic acid molecule consisting of the nucleic acid sequence set forth in SEQ ID NO:2 under stringent conditions. The nucleic acid molecule consisting of the nucleic acid sequence set forth in SEQ ID NO:2 encodes a tomato prosystemin molecule. As described in Examples 1 and 2 herein, the present inventors introduced a vector, including a tomato prosystemin cDNA molecule, under the control of a constitutive promoter, into potato plants. Tubers formed from these transgenic potato plants expressed high levels of proteinase inhibitor I, proteinase inhibitor II, and patatin, and had an elevated level of water-soluble protein. While not wishing to be bound by theory, it is believed that the prosystemin molecule is processed in the potato plant to yield systemin which induces synthesis of proteinase inhibitor I, proteinase inhibitor II, and patatin in the tubers.

Thus, in one aspect, the present invention provides potato tubers that each include water-soluble protein at a concentration of at least 5 mg/ml (such as at least 7 mg/ml, or such as at least 10 mg/ml, or such as at least 20 mg/ml), and that each include an exogenous nucleic acid molecule that (a) encodes a prosystemin molecule, and (b) hybridizes to the complement of the nucleic acid molecule consisting of the nucleic acid sequence set forth in SEQ ID NO:2 under conditions of 1×SSC at 50° C. for 30 minutes. Some potato tubers of this aspect of the invention include water-soluble protein at a concentration of at least 5 mg/ml (such as at least 7 mg/ml, or such as at least 10 mg/ml, or such as at least 20 mg/ml), and include an exogenous nucleic acid molecule that (a) encodes a prosystemin molecule, and (b) hybridizes to the complement of the nucleic acid molecule consisting of the nucleic acid sequence set forth in SEQ ID NO:2 under conditions of 0.5×SSC at 50° C. for 30 minutes. Some potato tubers of this aspect of the invention include water-soluble protein at a concentration of at least 5 mg/ml (such as at least 7 mg/ml, or such as at least 10 mg/ml, or such as at least 20 mg/ml), and include an exogenous nucleic acid molecule that (a) encodes a prosystemin molecule, and (b) hybridizes to the complement of the nucleic acid molecule consisting of the nucleic acid sequence set forth in SEQ ID NO:2 under conditions of 0.5×SSC at 60° C. for 30 minutes.

In another aspect, the present invention provides potato tubers that each include water-soluble protein at a concentration of at least 5 mg/ml (such as at least 7 mg/ml, or such as at least 10 mg/ml, or such as at least 20 mg/ml), proteinase inhibitor I at a concentration of at least 500 μg/ml (such as at least 750 μg/ml, or such as at least 1000 μg/ml), proteinase inhibitor II at a concentration of at least 800 μg/ml (such as at least 1000 μg/ml, or such as at least 1200 μg/ml), and that each include an exogenous nucleic acid molecule that (a) encodes a prosystemin molecule, and (b) hybridizes to the complement of the nucleic acid molecule consisting of the nucleic acid sequence set forth in SEQ ID NO:2 under conditions of 1×SSC at 50° C. for 30 minutes.

Some potato tubers of this aspect of the invention include water-soluble protein at a concentration of at least 5 mg/ml (such as at least 7 mg/ml, or such as at least 10 mg/ml, or such as at least 20 mg/ml), proteinase inhibitor I at a concentration of at least 500 μg/ml (such as at least 750 μg/ml, or such as at least 1000 μg/ml), proteinase inhibitor II at a concentration of at least 800 μg/ml (such as at least 1000 μg/ml, or such as at least 1200 μg/ml), and include an exogenous nucleic acid molecule that (a) encodes a prosystemin molecule, and (b) hybridizes to the complement of the nucleic acid molecule consisting of the nucleic acid sequence set forth in SEQ ID NO:2 under conditions of 0.5×SSC at 50° C. for 30 minutes.

Some potato tubers of this aspect of the invention include water-soluble protein at a concentration of at least 5 mg/ml (such as at least 7 mg/ml, or such as at least 10 mg/ml, or such as at least 20 mg/ml), proteinase inhibitor I at a concentration of at least 500 μg/ml (such as at least 750 μg/ml, or such as at least 1000 μg/ml), proteinase inhibitor II at a concentration of at least 800 μg/ml (such as at least 1000 μg/ml, or such as at least 1200 μg/ml), and include an exogenous nucleic acid molecule that (a) encodes a prosystemin molecule, and (b) hybridizes to the complement of the nucleic acid molecule consisting of the nucleic acid sequence set forth in SEQ ID NO:2 under conditions of 0.5×SSC at 60° C. for 30 minutes.

In another aspect, the present invention provides potato tubers that each include an exogenous nucleic acid molecule that (a) encodes a systemin molecule, and (b) hybridizes to a nucleic acid molecule consisting of the nucleic acid sequence set forth in SEQ ID NO:4 under conditions of 5×SSC at 55° C. for 30 minutes. Some potato tubers of this aspect of the invention include an exogenous nucleic acid molecule that (a) encodes a systemin molecule, and (b) hybridizes to a nucleic acid molecule consisting of the nucleic acid sequence set forth in SEQ ID NO:4 under conditions of 5×SSC at 60° C. for 30 minutes.

In another aspect, the present invention provides potato tubers that each include an exogenous nucleic acid molecule that (a) encodes a systemin molecule, and (b) hybridizes to the complement of the nucleic acid molecule consisting of the nucleic acid sequence set forth in SEQ ID NO:2 under conditions of 1×SSC at 50° C. for 30 minutes. Some potato tubers of this aspect of the invention include an exogenous nucleic acid molecule that (a) encodes a systemin molecule, and (b) hybridizes to the complement of the nucleic acid molecule consisting of the nucleic acid sequence set forth in SEQ ID NO:2 under conditions of 0.5×SSC at 50° C. for 30 minutes. Some potato tubers of this aspect of the invention include an exogenous nucleic acid molecule that (a) encodes a systemin molecule, and (b) hybridizes to the complement of the nucleic acid molecule consisting of the nucleic acid sequence set forth in SEQ ID NO:2 under conditions of 0.5×SSC at 60° C. for 30 minutes.

In another aspect, the present invention provides potato tubers that each include an exogenous nucleic acid molecule that (a) encodes a prosystemin molecule, and (b) hybridizes to the complement of the nucleic acid molecule consisting of the nucleic acid sequence set forth in SEQ ID NO:2 under conditions of 1×SSC at 50° C. for 30 minutes. Some potato tubers of this aspect of the invention include an exogenous nucleic acid molecule that (a) encodes a prosystemin molecule, and (b) hybridizes to the complement of the nucleic acid molecule consisting of the nucleic acid sequence set forth in SEQ ID NO:2 under conditions of 0.5×SSC at 50° C. for 30 minutes. Some potato tubers of this aspect of the invention include an exogenous nucleic acid molecule that (a) encodes a prosystemin molecule, and (b) hybridizes to the complement of the nucleic acid molecule consisting of the nucleic acid sequence set forth in SEQ ID NO:2 under conditions of 0.5×SSC at 60° C. for 30 minutes.

Hybridization can be conducted, for example, by utilizing the technique of hybridizing radiolabelled nucleic acid probes to nucleic acid molecules immobilized on nitrocellulose filters or nylon membranes as set forth at pages 9.52 to 9.55 of Molecular Cloning, A Laboratory Manual (2nd edition), J. Sambrook, E. F. Fritsch and T. Maniatis eds., the cited pages of which are incorporated herein by reference.

Thus, for example, to determine if a nucleic acid molecule that encodes prosystemin (the probe nucleic acid molecule) hybridizes under conditions of 1×SSC at 50° C. for 30 minutes to the complement of the nucleic acid molecule consisting of the nucleic acid sequence set forth in SEQ ID NO:2 (the target nucleic acid molecule), the target nucleic acid molecule is transferred to a nitrocellulose or nylon membrane and hybridized in the presence of the radiolabelled probe nucleic acid molecule. The concentration of the SSC in the hybridization solution is no less than 1×SSC, and the hybridization temperature is no greater than 50° C. The membrane is then washed under conditions of 1×SSC at 50° C. for 30 minutes (optionally the membrane is first washed under conditions that are less stringent than 1×SSC at 50° C. for 30 minutes, before being washed under conditions of 1×SSC at 50° C. for 30 minutes). If the probe nucleic acid molecule hybridizes to the target nucleic acid molecule, under conditions of 1×SSC at 50° C. for 30 minutes, then the radioactive probe nucleic acid molecule will remain hybridized to the target nucleic acid molecule during washing at 1×SSC at 50° C. for 30 minutes, and the location of the hybridized, radioactive, probe nucleic acid molecule on the membrane will appear as a dark band when the filter is exposed to a photographic film.

Nucleic acid molecules that encode a systemin molecule, or a prosystemin molecule, can be identified and isolated by using any art-recognized technique for identifying and isolating nucleic acid molecules. For example, the nucleic acid molecule having the sequence set forth in SEQ ID NO:2, can be used as a hybridization probe to screen a plant genomic or cDNA library. The technique of hybridizing radiolabelled nucleic acid probes to nucleic acids immobilized on nitrocellulose filters or nylon membranes can be used to screen the genomic or cDNA library to identify nucleic acid molecules that encode a systemin molecule or a prosystemin molecule. Typically, hybridization conditions are no more than 25° C. to 30° C. (for example, 10° C.) below the melting temperature (Tm) of the native duplex. Tm for nucleic acid molecules greater than about 100 bases can be calculated by the formula Tm=81.5+0.41% (G+C)-log(Na⁺). For oligonucleotide molecules less than 100 bases in length, exemplary hybridization conditions are 5 to 10° C. below Tm. On average, the Tm of a short oligonucleotide duplex is reduced by approximately (500/oligonucleotide length)° C.

Again, by way of example, nucleic acid molecules that encode a systemin molecule, or a prosystemin molecule, can be isolated by the polymerase chain reaction (PCR) described in The Polymerase Chain Reaction (K. B. Mullis et al., eds. 1994), incorporated herein by reference.

Nucleic acid molecules that encode a systemin molecule, or a prosystemin molecule, can also be synthesized by any art-recognized means, e.g., by use of an automated DNA synthesizer (such as are commercially available from Biosearch, Applied Biosystems, etc.) and standard phosphoramidite chemistry. As examples, phosphorothioate oligonucleotides may be synthesized by the method of Stein et al. (Nucl. Acids Res. 16:3209-3221, 1988), methylphosphonate oligonucleotides can be prepared by use of controlled pore glass polymer supports (Sarin et al., 1988, Proc. Natl. Acad. Sci. U.S.A. 85:7448-7451).

Non-limiting examples of useful nucleic acid molecules that encode a prosystemin molecule are set forth in McGurl et al., Science 255: 1570-1573 (1992), and in C. P. Constabel, et al., Plant Mol. Biol., 34: 55-62 (1998), which publications are incorporated herein by reference.

Exogenous nucleic acid molecules, that encode a systemin or prosystemin molecule, are typically introduced into a potato plant as part of a vector. Thus, nucleic acid molecules that encode a systemin or prosystemin molecule are typically present within potato tubers of the invention as part of a vector. The term “vector” refers to a nucleic acid molecule, usually double-stranded DNA, which may have inserted into it another nucleic acid molecule (the insert nucleic acid molecule), such as a nucleic acid molecule that encodes a systemin or prosystemin molecule. The vector is used to transport the insert nucleic acid molecule into a suitable host cell, such as a cell of a potato plant. A vector may contain the necessary elements that permit transcribing the insert nucleic acid molecule, and, optionally, translating the transcript into a polypeptide. The insert nucleic acid molecule (e.g., a nucleic acid molecule that encodes a systemin or prosystemin molecule) may be derived from the host cell, or may be derived from a different cell or organism. Once in the host cell, the vector can replicate independently of, or coincidental with, the host chromosomal DNA, and several copies of the vector and its inserted nucleic acid molecule may be generated. The term “vector” includes the T-DNA of a Ti plasmid. The following publications describe representative vectors that are useful in the practice of the present invention: An, G. et al., Binary Vectors, In Plant Molecular Biology Manual A3, Klewer Academic Publishers, Dordrech, Belgium; McBride, K. E., and Summerfeld, K. R., Plant Mol. Biol. 14: 369-376 (1990), which publications are incorporated herein by reference.

Thus, in one aspect, the present invention provides potato tubers that each include a vector that includes a nucleic acid molecule that encodes a systemin or prosystemin molecule. Typically the vector includes elements required to express the encoded systemin or prosystemin molecule. Any of the exogenous nucleic acid molecules, that encode a systemin or prosystemin molecule, described herein can be incorporated into a vector and introduced into a potato tuber in accordance with the present invention. Some potato tubers of the invention include (a) a vector that includes a nucleic acid molecule that encodes a systemin or prosystemin molecule, and (b) water-soluble protein at a concentration of at least 5 mg/ml (such as at least 7 mg/ml, or such as at least 10 mg/ml, or such as at least 20 mg/ml). Some potato tubers of the invention include (a) a vector that includes a nucleic acid molecule that encodes a systemin or prosystemin molecule, (b) water-soluble protein at a concentration of at least 5 mg/ml (such as at least 7 mg/ml, or such as at least 10 mg/ml, or such as at least 20 mg/ml); proteinase inhibitor I at a concentration of at least 500 μg/ml (such as at least 750 μg/ml, or such as at least 1000 μg/ml), and proteinase inhibitor II at a concentration of at least 800 μg/ml (such as at least 1000 μg/ml, or such as at least 1200 μg/ml).

Potato tubers of the invention that include an exogenous nucleic acid molecule that encodes a systemin molecule, or a prosystemin molecule, can be prepared by any art-recognized means for introducing nucleic acid molecules into plants. These methods include, but are not limited to, (1) direct DNA uptake, such as particle bombardment or electroporation (see, Klein et al., Nature 327:70-73 (1987); U.S. Pat. No. 4,945,050), and (2)Agrobacterium-mediated transformation (see, e.g., U.S. Pat. Nos. 6,051,757; 5,731,179; 4,693,976; 4,940,838; 5,464,763; and 5,149,645). Within the cell, the exogenous nucleic acid molecule may be incorporated within a chromosome.

Transgenic plants can be obtained, for example, by transferring vectors that include a selectable marker gene, e.g., the kan gene encoding resistance to kanamycin, into Agrobacterium tumifaciens containing a helper Ti plasmid as described in Hoeckema et al., Nature, 303:179-181 (1983), and culturing the Agrobacterium cells with leaf slices, or other tissues or cells, of the plant to be transformed as described by An et al., Plant Physiology, 81:301-305 (1986).

Transformed plant calli may be selected through the selectable marker by growing the cells on a medium containing, for example, kanamycin, and appropriate amounts of phytohormone, such as naphthalene acetic acid and benzyladenine for callus and shoot induction. The plant cells may then be regenerated and the resulting plants transferred to soil using techniques well known to those skilled in the art.

In addition to the methods described above, several methods are known in the art for transferring DNA and vectors into a wide variety of plant species, including gymnosperms, angiosperms, monocots and dicots (see, e.g., Glick and Thompson, eds., Methods in Plant Molecular Biology, CRC Press, Boca Raton, Fla. (1993)). An example includes treatment of protoplasts with polyethylene glycol (see, e.g., Lyznik et al., Plant Molecular Biology, 13:151-161 (1989)). Further, plant viruses can be used as vectors to transfer nucleic acid molecules into plant cells. Examples of plant viruses that can be used as vectors to transform plants include the Cauliflower Mosaic Virus (see, e.g., Brisson et al., Nature 310:511-514 (1984). Other useful techniques include: site-specific recombination using the Cre-lox system (see, U.S. Pat. No. 5,635,381); and insertion into a target sequence by homologous recombination (see, U.S. Pat. No. 5,501,967). Additionally, plant transformation strategies and techniques are reviewed in Birch, R. G., Ann Rev Plant Phys Plant Mol. Biol., 48:297 (1997); and in Forester et al., Exp. Agric., 33:15-33 (1997). An exemplary method for introducing an exogenous nucleic acid molecule into potato plants is set forth in Kumar, A., et al., Plant Journal. 9:821-829 (1996).

The cells which have been transformed may be grown into plants by a variety of art-recognized means. See, for example, McConnick et al., Plant Cell Reports 5:81-84 (1986). These plants may then be grown, and either selfed or crossed with a different plant strain, and the resulting homozygotes or hybrids having the desired phenotypic characteristic identified. Two or more generations may be grown to ensure that the subject phenotypic characteristic is stably maintained and inherited, and then seeds may be harvested to ensure the desired phenotype or other property has been achieved.

In another aspect, the present invention provides methods for increasing the amount of water-soluble protein in a potato tuber. The methods of this aspect of the invention each include the step of expressing an exogenous systemin molecule in the potato tuber, whereby the synthesis of at least one water-soluble protein is induced by the exogenous systemin molecule. By way of example, any of the systemin molecules disclosed in the present patent application are useful in the methods of the invention. The exogenous systemin molecule can be expressed as a mature eighteen amino acid polypeptide, or can be expressed as a larger precursor protein, which is processed within a potato cell to yield a mature, eighteen amino acid, systemin polypeptide.

Thus, in some embodiments, the present invention provides methods for increasing the amount of water-soluble protein in a potato tuber, the methods of these embodiments each including the step of expressing an exogenous systemin molecule in the potato tuber, whereby the synthesis of at least one water-soluble protein is induced by the exogenous systemin molecule, and wherein the systemin molecule is encoded by a nucleic acid molecule that hybridizes under conditions of 5×SSC at 55° C. for 30 minutes to a nucleic acid molecule consisting of the nucleic acid sequence set forth in SEQ ID NO:4. In other embodiments, the present invention provides methods for increasing the amount of water-soluble protein in a potato tuber, the methods each including the step of expressing an exogenous systemin molecule in the potato tuber, whereby the synthesis of at least one water-soluble protein is induced by the exogenous systemin molecule, and wherein the systemin molecule is encoded by a nucleic acid molecule that hybridizes under conditions of 5×SSC at 60° C. for 30 minutes to a nucleic acid molecule consisting of the nucleic acid sequence set forth in SEQ ID NO:4. The nucleic acid sequence set forth in SEQ ID NO:4 is the sequence of the complement of the portion of the tomato prosystemin molecule, set forth in SEQ ID NO:2, that encodes the mature systemin polypeptide (SEQ ID NO:1).

In other embodiments, the present invention provides methods for increasing the amount of water-soluble protein in a potato tuber, the methods of these embodiments each including the step of expressing an exogenous systemin molecule in the potato tuber, whereby the synthesis of at least one water-soluble protein (such as proteinase inhibitor I and/or proteinase inhibitor II) is induced by the exogenous systemin molecule, and wherein the systemin molecule is encoded by a nucleic acid molecule that hybridizes under conditions of 1×SSC at 50° C. for 30 minutes to the complement of a nucleic acid molecule consisting of the nucleic acid sequence set forth in SEQ ID NO:2. In other embodiments, the present invention provides methods for increasing the amount of water-soluble protein in a potato tuber, the methods each including the step of expressing an exogenous systemin molecule in the potato tuber, whereby the synthesis of at least one water-soluble protein is induced by the exogenous systemin molecule, and wherein the systemin molecule is encoded by a nucleic acid molecule that hybridizes under conditions of 0.5×SSC at 50° C. for 30 minutes to the complement of a nucleic acid molecule consisting of the nucleic acid sequence set forth in SEQ ID NO:2. In other embodiments, the present invention provides methods for increasing the amount of water-soluble protein in a potato tuber, the methods each including the step of expressing an exogenous systemin molecule in the potato tuber, whereby the synthesis of at least one water-soluble protein is induced by the exogenous systemin molecule, and wherein the systemin molecule is encoded by a nucleic acid molecule that hybridizes under conditions of 0.5×SSC at 60° C. for 30 minutes to the complement of a nucleic acid molecule consisting of the nucleic acid sequence set forth in SEQ ID NO:2. The nucleic acid sequence set forth in SEQ ID NO:2 is the sequence of a tomato prosystemin molecule.

In some embodiments, the present invention provides methods for increasing the amount of water-soluble protein in a potato tuber, the methods of these embodiments each including the step of expressing an exogenous systemin molecule in the potato tuber, whereby the synthesis of at least one water-soluble protein (such as proteinase inhibitor I and/or proteinase inhibitor II) is induced by the exogenous systemin molecule, and wherein the amount of water-soluble protein in the potato tuber expressing the exogenous systemin molecule is at least 5 mg/ml (such as at least 7 mg/ml, or such as at least 10 mg/ml, or such as at least 20 mg/ml). Thus, in some embodiments of the methods of this aspect of the invention, the synthesis of both proteinase inhibitor I and proteinase inhibitor II are induced by the exogenous systemin molecule. For example, in some embodiments of the methods' of this aspect of the invention, the amount of proteinase inhibitor I in the potato tuber expressing an exogenous systemin molecule is at least 500 μg/ml (such as at least 750 μg/ml, or such as at least 1000 μg/ml), and the amount of proteinase inhibitor II in the potato tuber expressing an exogenous systemin molecule is at least 800 μg/ml (such as at least 1000 μg/ml, or such as at least 1200 g/ml).

The present invention also provides processed potato products, such as food products, prepared from the potato tubers of the invention. Representative examples of the processed potato products of the invention include potato chips and French fries.

The present invention also provides populations of potato tubers, such as populations of at least 10 potato tubers, or such as populations of at least 100 potato tubers, or such as populations of at least 1000 potato tubers. The populations of potato tubers can be formed from any of the potato tubers of the invention.

The following examples merely illustrate the best mode now contemplated for practicing the invention, but should not be construed to limit the invention.

### EXAMPLE 1

This example describes the preparation of transgenic potato plants that express an exogenous nucleic acid molecule that encodes prosystemin (hereinafter referred to as the prosystemin transgene).

Preparation of prosystemin transgene: an exogenous nucleic acid molecule that encodes a tomato prosystemin (SEQ ID NO:3) was prepared as described in McGurl et al, Proc. Nat'l. Acad. Sci. U.S.A., 91: 9799-9802 (1994). The 5′ end of an 800-bp fragment of a prosystemin cDNA (McGurl et al., Science 255: 1570-1573 (1992)), encoding amino acids 16-200 of prosystemin (SEQ ID NO:3), was ligated to a double-stranded oligonucleotide encoding the first 15 amino acids of prosystemin plus 15 bases of the 5′ untranslated region. The resulting fragment was inserted into the polylinker of pBlue-script KS (+) (Stratagene), and DNA derived from a single recombinant was digested with HindIII (5′ end) and Ssp I (3′ end), generating a cDNA fragment which encoded the complete prosystemin open reading frame (SEQ ID NO:3) plus 15 bp of the 5′ untranslated region and 103 bp of the 3′ untranslated region. This fragment was cloned in the sense orientation relative to the ³⁵S cauliflower mosaic virus (CaMV) promoter within the binary vector pGA 643, which had been digested with Hind III and Hpa I.

Potato plant material transformed with prosystemin transgene: Tuber discs or leaf segments of potato c.v. Désirée were transformed with the prosystemin transgene.

Sterilization of plant material: Tubers were peeled, washed briefly in distilled water and surface-sterilized for 15 minutes in a 10% solution of sodium hypochlorite (commercial bleach) containing a few drops of Tween 20. The tubers were then washed six times with sterile distilled water and finally immersed in liquid MS medium.

Leaves were sterilized for 10 minutes in a 10% solution of sodium hypochlorite containing a few drops of Tween 20. Then, the leaves were washed six times in sterile distilled water.

Explant preparation: After sterilization, columnar sections of potato tubers were obtained using a sterile, 1 cm-diameter, cork borer, and then the columnar sections were sliced into thin discs (approximately 2-3 mm thick) using a scalpel. After sterilization, the leaves were cut into small squares (1-2 cm²), then the squares were perforated with forceps or syringe needles.

Preparation of tobacco feeder plates: Tobacco suspension cells (Line NT-1) were subcultured to fresh medium number 1, and incubated for four days under dark conditions at 25° C. Then they were plated on solid potato medium number 2, adding 3 ml of fresh tobacco cell suspension per Petri dish. The cells were incubated under dark conditions at 25° C. for another two days.

A sterile filter paper (Whatman), presoaked in liquid potato medium number 2, was placed in each Petri dish over the fine layer of tobacco cells that were spread over the whole surface of the potato solid medium. Then, the tobacco feeder plates were ready for the preculture of the potato tuber and leaf explants.

The compositions of medium number 1 and medium number 2 are set forth in Table 1 below.

TABLE 1
Medium No. 1 for NT-1 tobacco suspension cells.

| For 1 liter of medium: | |
|---|---|
| MS Salts (Gibco) | 4.3 grams |
| Sucrose | 30 grams |
| Inositol | 100 mg |
| Thiamine | 1 mg |
| KH₂PO₄ | 3 ml of millers I (millers I = 60 gr/L) |
| 2,4-D | 0.2 mg/L |
| pH | 5.7-5.8 |

Medium No. 2 for potato (callus and shoot induction)

| | |
|---|---|
| MS Salts (Gibco) | 4.3 grams |
| Inositol | 100 mg |
| Sucrose | 30 grams |
| B5 Vitamins | 1 ml (1000 × stock) |
| Zeatin | 5 μM (or 1 ml of 1 mg/ml stock) |
| IAA | 3 μM(or 0.5 ml of 1 mg/ml stock) |
| pH | 5.9 |
| Agarose | 7 grams |

Cefotaxime (preculture non-selective and selective medium): 250 mg/L
Kanamycin (selective medium): 100 mg/L.

Pre-conditioning on feeder plates: Leaf squares and potato discs were conditioned on feeder plates for two days before their treatment with Agrobacterium.

Treatment with Agrobacterium (cocultivation): Preconditioned explants were infected with Agrobacterium by soaking them, for 20-30 minutes, in 10-20 ml of liquid potato medium containing 107 Agrobacterium cells/ml. Then the explants were blotted dry with sterile filter paper, and incubated again on the tobacco feeder plates for another two days.

Preculture in non-selective medium: After the cocultivation on the feeder plates, the explants were transferred to fresh-solid potato medium number 2 containing 250 mg/L of Cefotaxime.

Culture in selective medium: When small calli (and sometimes incipient shoots) arose from the explants after 2-3 weeks, the explants were transferred to the same medium but also including 100 mg/L of kanamycin. The explants were subcultured to fresh medium every two weeks (growing calli were separated from the rest of the explant), until the first well-differentiated shoots appeared (after 4-6 weeks). After that, the level of kanamycin in the medium was gradually reduced, first to 50 mg/L and then to 20 mg/L, to allow the formation of whole plants.

### EXAMPLE 2

This example describes the characterization of the water-soluble protein levels, and the levels of proteinase inhibitor I and proteinase inhibitor II, in the leaves and tubers of the transgenic potato plants prepared as described in Example 1 herein.

Eighteen transgenic potato plants were recovered from the transformation experiment described in Example 1 and planted in the greenhouse. The levels of proteinase Inhibitor I and proteinase Inhibitor II were assayed by radial immunodiffusion using pure potato Inhibitor I and II as standards in leaves of all eighteen transgenic lines. The radial immunodiffusion method is described in Ryan, C. A., Anal. Biochem. 19: 434-440 (1967), and in Example 3 herein. The levels of proteinase Inhibitor I and proteinase Inhibitor II in the leaves of the transgenic potato plants are shown in Table 2 (values are the average values of two measurements per plant; each measurement was made using pooled juice from at least two leaves). Two types of control potato plants were also generated and analyzed: a wild-type control which did not include any transgene or vector; and a transgenic control that included the same vector (but without a prosystemin transgene insert) used to generate the transgenic potato plants.

TABLE 2

| | Inhibitor I | Inhibitor II |
|---|---|---|
| Plant | (μg/ml leaf juice) | (μg/ml leaf juice) |
| Wild Type Control | 44 | 0 |
| Transgenic Control | 23 | 20 |
| Transgenic (T) #1 | 24 | 30 |
| T #2 | 75 | 77 |
| T #3 | 50 | 17 |
| T #4 | 80 | 90 |
| T #5 | 53 | 87 |
| T #6 | 110 | 100 |
| T #7 | 32 | 68 |
| T #8 | 25 | 70 |
| T #9 | 11 | 125 |
| T #10 | 92 | 42 |
| T #11 | 100 | 80 |
| T #12 | 88 | 74 |
| T #13 | 77 | 77 |
| T #14 | 94 | 148 |
| T #15 | 94 | 5 |
| T #16 | 36 | 87 |
| T #17 | 44 | 60 |
| T #18 | 57 | 42 |

As can be seen from the data presented in Table 2, several transgenic potato plants exhibited elevated levels of Inhibitor I and II over controls. Prosystemin mRNA levels were then assayed in leaves using Northern blot analysis, and confirmed that in transgenic potato plants having elevated levels of Inhibitor I and II protein in leaves, elevated levels of prosystemin mRNA were also present in leaves.

Seven transgenic potato plants having elevated levels of Inhibitor I and Inhibitor II in the leaves (transformed plants #1, #2, #3, #4, #6, #10, #14 and #5) were grown in large pots in the greenhouse, supplemented with lights, where they set tubers. To determine if the expression of the prosystemin transgene affected levels of water-soluble proteins in potato tubers, the levels of Inhibitor I and II were assayed by radial immunodiffusion in juice expressed from tubers. Nineteen randomly selected tubers were collected from the transgenic plants, and five tubers from control plants, with tuber sizes ranging from 5 grams to 30 grams. A transverse section (about one quarter inch in width) was excised from the center of each tuber, including cortical and pith tissue. The section was diced into small pieces and crushed with mortar and pestle to express the juice. The juice was collected in a microfuge tube and centrifuged at 8000 rpm to clarify. The Bradford assay was used to measure the amount of water-soluble protein in the clarified juice. The amounts of proteinase inhibitor I and proteinase inhibitor II were measured using immunodiffusion. Centrifugation had no effect on the levels of Inhibitor I and II in the juice.

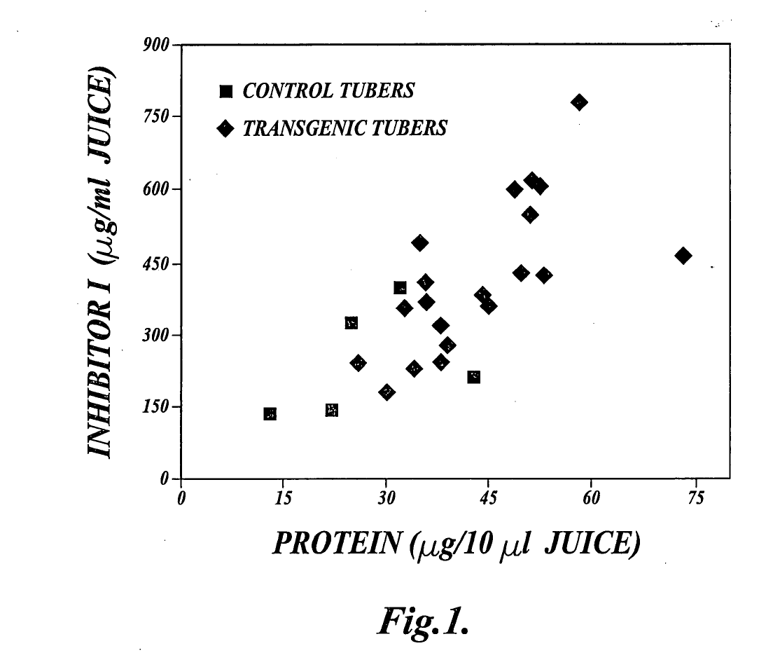

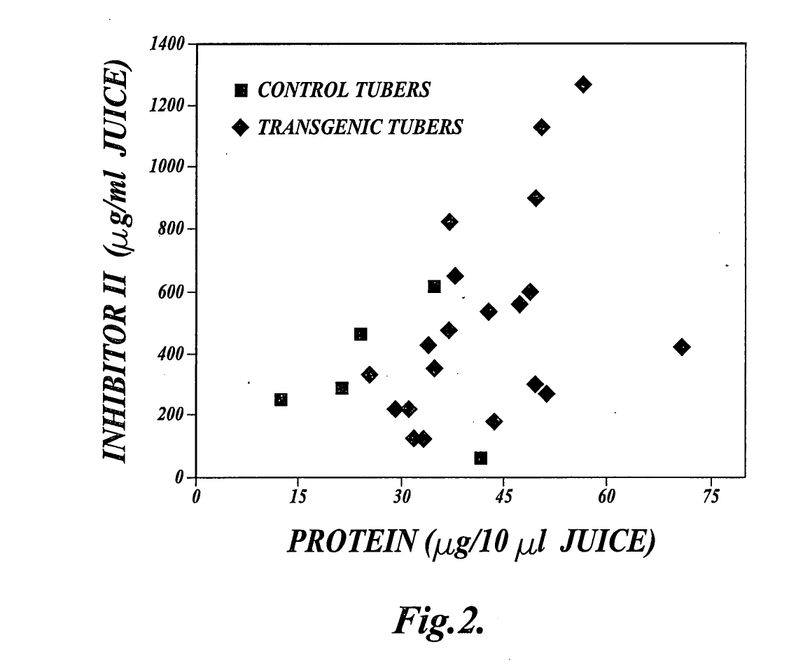

The results, shown in FIG. 1 and FIG. 2, show that tubers from several of the transgenic potato plants exhibited an overall large increase in the amounts of both Inhibitor I and II proteins, and in the amount of total soluble proteins. The average Inhibitor I and II levels in the transgenic (transformed plants #1, #2, #3, #4, #6, #10, #14 and #5) and wild-type control tubers is compared in Table 3 below. Bovine serum albumin was used as a concentration standard to determine the amount of water-soluble protein.

TABLE 3
Average Proteinase Inhibitor I and II and Soluble Protein Levels
in Wild-Type and Transgenic Potato Tubers

| | |
|---|---|
| Proteinase Inhibitor Levels | Soluble Protein |
| (μg/ml tuber juice) | (μg in 10 μl tuber |

| | Inhibitor I | Inhibitor II | juice) |
|---|---|---|---|
| Wild-Type | 234 | 330 | 28 |
| Transgenics | 486 | 653 | 55 |
| % Increase | 207% | 198% | 196% |

SDS-polyacrylamide gel electrophoresis of juice from tubers of transgenic plant numbers #2, #3, #6, #14 and #15 showed that the levels of Inhibitor I, Inhibitor II and patatin (a major storage protein of potatoes), are increased over the wild-type level in each of the transgenic tubers. The amount of increase in the amount of individual proteins differs between individual transformants, likely because of positional effects and somaclonal variations that occur during regeneration of transgenic plants from single cells.

### EXAMPLE 3

This Example describes an immunodiffusion method for measuring the amounts of proteinase inhibitor I and proteinase Inhibitor II in juice expressed from potato tubers. The method is based on the method disclosed by C. A. Ryan, Analytical Biochemistry, 19: 434-440 (1967), which publication is incorporated herein by reference.

Immunodiffusion plates are prepared as follows. Two grams of Noble Agar (Difco Co., Detroit, Mich.) are dissolved in 100 ml buffer (20 mM Tris, 0.9% NaCl, pH 8.5) by bringing the buffer, including agar, to a boil. The dissolved agar is cooled to 58° C. and rabbit antiserum (including either antibodies raised against potato proteinase inhibitor I, or antibodies raised against tomato proteinase inhibitor II) is added to the agar solution in an amount of from 2 ml antiserum per 100 ml agar solution, to 5 ml antiserum per 100 ml agar solution, depending on the strength of the antiserum. Eleven milliliters of the agar solution, including antiserum, are pipetted into a square Petri dish (Falcon #351012, Fisher Scientific, Houston, Tex.). After the agar has set in the Petri dish, 0.25 cm diameter holes are punched in the agar and the agar plugs are removed from the holes.

Potato tuber juice is prepared as follows: a transverse section (about one quarter inch in width) is excised from the center of the tuber, including cortical and pith tissue. The section is diced into small pieces and crushed with mortar and pestle to express the juice. The juice is collected in a microfuge tube and centrifuged at 8000 rpm to clarify.

A standard curve is constructed using potato proteinase inhibitor I and potato proteinase inhibitor II in a dilution series of from 1 mg/ml to 0.125 mg/ml. The diluted inhibitors I and II are plated in wells in agar plates including anti-inhibitor I and anti-inhibitor II antibodies, respectively, and incubated for 24 hours. The plates are then developed using 7.5% acetic acid.

The amount of proteinase inhibitor I and proteinase inhibitor II in potato tuber juice is quantitatedx using the standard curve.

While the preferred embodiment of the invention has been illustrated and described, it will be appreciated that various changes can be made therein without departing from the spirit and scope of the invention.

1. A potato tuber comprising water-soluble protein at a concentration of at least 5 mg/ml.

2. A potato tuber of claim 1 comprising water-soluble protein at a concentration of at least 7 mg/ml.

3. A potato tuber of claim 1 comprising water-soluble protein at a concentration of at least 10 mg/ml.

4. A potato tuber of claim 1 comprising water-soluble protein at a concentration of at least 20 mg/ml.

5. A potato tuber of claim 1 comprising proteinase Inhibitor I at a concentration of at least 500 μg/ml, and proteinase Inhibitor II at a concentration of at least 800 μg/ml.

6. A potato tuber of claim 1 comprising proteinase Inhibitor I at a concentration of at least 750 μg/ml, and proteinase Inhibitor II at a concentration of at least 1000 μg/ml.

7. A potato tuber of claim 1 comprising proteinase Inhibitor I at a concentration of at least 1000 μg/ml, and proteinase Inhibitor II at a concentration of at least 1200 μg/ml.

8. A potato tuber of claim 1 comprising an exogenous nucleic acid molecule that encodes a systemin molecule, said nucleic acid molecule hybridizing to a nucleic acid molecule consisting of the sequence set forth in SEQ ID NO:4 under conditions of 5×SSC at 55° C. for 30 minutes.

9. A potato tuber of claim 8 wherein said systemin molecule consists of the amino acid sequence set forth in SEQ ID NO:1.

10. A potato tuber of claim 1 comprising an exogenous nucleic acid molecule that encodes a systemin molecule, said nucleic acid molecule hybridizing to the complement of a nucleic acid molecule consisting of the sequence set forth in SEQ ID NO:2 under conditions of 1×SSC at 50° C. for 30 minutes.

11. A potato tuber of claim 1 comprising an exogenous nucleic acid molecule that encodes a systemin molecule, said nucleic acid molecule hybridizing to the complement of a nucleic acid molecule consisting of the sequence set forth in SEQ ID NO:2 under conditions of 0.5×SSC at 50° C. for 30 minutes.

12. A potato tuber of claim 10 wherein said systemin molecule consists of the amino acid sequence set forth in SEQ ID NO:1.

13. A potato tuber of claim 1 comprising an exogenous nucleic acid molecule that encodes a prosystemin molecule, said nucleic acid molecule hybridizing to the complement of the nucleic acid molecule consisting of the sequence set forth in SEQ ID NO:2 under conditions of 1×SSC at 50° C. for 30 minutes.

14. A potato tuber of claim 1 comprising an exogenous nucleic acid molecule that encodes a prosystemin molecule, said nucleic acid molecule hybridizing to the complement of the nucleic acid molecule consisting of the sequence set forth in SEQ ID NO:2 under conditions of 0.5×SSC at 50° C. for 30 minutes.

15. A potato tuber of claim 13 wherein said prosystemin molecule consists of the amino acid sequence set forth in SEQ ID NO:3.

16. A potato tuber comprising an exogenous nucleic acid molecule that encodes a systemin molecule, said nucleic acid molecule hybridizing under conditions of 5×SSC at 55° C. for 30 minutes to a nucleic acid molecule consisting of the nucleic acid sequence set forth in SEQ ID NO:4.

17. A potato tuber comprising an exogenous nucleic acid molecule that encodes a systemin molecule, said nucleic acid molecule hybridizing to the complement of a nucleic acid molecule consisting of the sequence set forth in SEQ ID NO:2 under conditions of 1×SSC at 50° C. for 30 minutes.

18. A potato tuber of claim 17 wherein said nucleic acid molecule hybridizes to the complement of a nucleic acid molecule consisting of the sequence set forth in SEQ ID NO:2 under conditions of 0.5×SSC at 50° C. for 30 minutes.

19. A potato tuber comprising an exogenous nucleic acid molecule that encodes a prosystemin molecule, said nucleic acid molecule hybridizing under conditions of 1×SSC at 50° C. for 30 minutes to the complement of a nucleic acid molecule consisting of the nucleic acid sequence set forth in SEQ ID NO:2.

20. A potato tuber of claim 19 wherein said nucleic acid molecule hybridizes under conditions of 0.5×SSC at 50° C. for 30 minutes to the complement of a nucleic acid molecule consisting of the nucleic acid sequence set forth in SEQ ID NO:2.

21. A method of increasing the amount of water-soluble protein in a potato tuber, the method comprising the step of expressing an exogenous systemin molecule in a potato tuber, whereby the synthesis of at least one water-soluble protein is induced by the exogenous systemin molecule.

22. The method of claim 21 wherein said systemin molecule is at least 70% identical to a systemin molecule consisting of the amino acid sequence set forth in SEQ ID NO:1.

23. The method of claim 21 wherein said systemin molecule is at least 80% identical to a systemin molecule consisting of the amino acid sequence set forth in SEQ ID NO:1.

24. The method of claim 21 wherein said systemin molecule is at least 90% identical to a systemin molecule consisting of the amino acid sequence set forth in SEQ ID NO:1.

25. The method of claim 21 wherein said systemin molecule is at least 95% identical to a systemin molecule consisting of the amino acid sequence set forth in SEQ ID NO:1.

26. The method of claim 21 wherein the systemin molecule consists of an amino acid sequence selected from the group of amino acid sequences consisting of SEQ ID NO:1, SEQ ID NO:5, and SEQ ID NO:6.

27. The method of claim 21 wherein the systemin molecule consists of the amino acid sequence set forth in SEQ ID NO:1.

28. The method of claim 21 wherein the systemin molecule is encoded by an exogenous nucleic acid molecule that hybridizes under conditions of 5×SSC at 55° C. for 30 minutes to a nucleic acid molecule consisting of the nucleic acid sequence set forth in SEQ ID NO:4.

29. The method of claim 21 wherein the systemin molecule is encoded by an exogenous nucleic acid molecule that hybridizes under conditions of 1×SSC at 50° C. for 30 minutes to the complement of a nucleic acid molecule consisting of the nucleic acid sequence set forth in SEQ ID NO:2.

30. The method of claim 21 wherein the systemin molecule is encoded by an exogenous nucleic acid molecule that hybridizes under conditions of 0.5×SSC at 50° C. for 30 minutes to the complement of a nucleic acid molecule consisting of the nucleic acid sequence set forth in SEQ ID NO:2.

31. The method of claim 21 wherein the amount of water-soluble protein in the potato tuber treated in accordance with the method of claim 21 is at least 5 mg/ml.

32. The method of claim 21 wherein the amount of water-soluble protein in the potato tuber treated in accordance with the method of claim 21 is at least 7 mg/ml.

33. The method of claim 21 wherein the amount of water-soluble protein in the potato tuber treated in accordance with the method of claim 21 is at least 10 mg/ml.

34. The method of claim 21 wherein the amount of water-soluble protein in the potato tuber treated in accordance with the method of claim 21 is at least 20 mg/ml.

35. The method of claim 21 wherein the synthesis of proteinase inhibitor I and proteinase inhibitor II is induced by the exogenous systemin molecule.

36. The method of claim 35 wherein the amount of proteinase inhibitor I in the potato tuber is at least 500 μg/ml, and the amount of proteinase inhibitor II in the potato tuber is at least 800 μg/ml.

37. The method of claim 35 wherein the amount of proteinase inhibitor I in the potato tuber is at least 750 μg/ml, and the amount of proteinase inhibitor II in the potato tuber is at least 1000 μg/ml.

38. The method of claim 35 wherein the amount of proteinase inhibitor I in the potato tuber is at least 1000 μg/ml, and the amount of proteinase inhibitor II in the potato tuber is at least 1200 μg/ml.


SEQUENCE LISTING (8 sequences)

SEQ ID NO: 1 - protein, 18 aa, Tomato Systemin
AVQSKPPSKRDPPKMQTD

SEQ ID NO: 2 - DNA, 951 bp, Lycopersicon esculentum
  CDS (105)..(704)
aaaattaaatttgatatttggtttaactcgattttccatgaacacccttagtgatgagtatataaagctcagctcatgaagagttgaaataaactaagaaaaccatgggaactccttcatatgatatcaaaaacaaaggagatgacatgcaagaagaaccaaaggtgaaacttcaccatgagaagggaggagatgaaaaggaaaaaataattgaaaaagagactccatcccaagatatcaacaacaaagataccatctcttcatatgttttaagagatgatacacaagaaataccaaagatggaacatgaggagggaggatatgtaaaggagaaaattgttgaaaaggagactatatcccaatatatcatcaagattgaaggagatgatgatgcacaagaaaaactaaaggttgagtatgaggaggaagaatatgaaaaagagaaaatagttgaaaaagagactccatcccaagatatcaacaacaaaggagatgatgcacaagaaaaaccaaaggtggaacatgaggaaggagatgacaaagagactccatcacaagatatcatcaagatggaaggggagggtgcactagaaataacaaaggtggtatgtgagaaaattatagtacgagaagatcttgctgttcaatcaaaacctccatcaaagcgtgatcctcccaaaatgcaaacagacaataataaactctagaaacatccaaaaaaaattaataaataaaaaattataattcagaacgataaagtaaaaattctgaatttgtctcccgttagaaaagtaacttcaaataaatatttgtctttctttgtattttcaaagtgtaatttggttattgtactttgagaagctttctttagattgttatgtacttgtattgcttcctttcttttggcttatttatataatataaataaaaaataaataaatatctaaagat

SEQ ID NO: 3 - protein, 200 aa, Lycopersicon esculentum
MGTPSYDIKNKGDDMQEEPKVKLHHEKGGDEKEKIIEKETPSQDINNKDTISSYVLRDDTQEIPKMEHEEGGYVKEKIVEKETISQYIIKIEGDDDAQEKLKVEYEEEEYEKEKIVEKETPSQDINNKGDDAQEKPKVEHEEGDDKETPSQDIIKMEGEGALEITKVVCEKIIVREDLAVQSKPPSKRDPPKMQTDNNKL

SEQ ID NO: 4 - DNA, 54 bp, Lycopersicon esculentum
gtctgtttgcattttgggaggatcacgctttgatggaggttttgattgaacagc

SEQ ID NO: 5 - protein, 18 aa, Potato Systemin
AVHSTPPSKRDPPKMQTD

SEQ ID NO: 6 - protein, 18 aa, Potato Systemin
AAHSTPPSKRDPPKMQTD

SEQ ID NO: 7 - protein, 18 aa, Nightshade Systemin
AVRSTPPPKRDPPKMQTD

SEQ ID NO: 8 - protein, 18 aa, Pepper Systemin
AVHSTPPSKRPPPKMQTD